(19) United States
(12) Patent Application Publication
(10) Pub. No.: US 2001/0049828 A1
(43) Pub. Date: Dec. 6, 2001

(54) Genetic system for controlling background expression of transgene products

(76) Inventors: Charles G. Orosz, Columbus, OH (US); Dongyuan Xia, Powell, OH (US); Gayle M. Gordillo, Columbus, OH (US)

Correspondence Address: CALFEE HALTER & GRISWOLD, LLP, 800 SUPERIOR AVENUE, SUITE 1400, CLEVELAND, OH, 44114, US

(21) Appl. No.: 09/547,489

(22) Filed: Apr. 12, 2000

(51) Int. Cl. A01K 67/00; A01K 67/027; C12N 15/74; C07H 21/04

(52) U.S. Cl. 800/8; 800/4; 800/21; 435/320.1; 536/23.1

(57) ABSTRACT

A method and system for controlling the expression of transgene products in specific tissues in a transgenic animal is provided. The method comprises: a) providing a fist transgenic parent animal whose genome comprises an F transgene comprising an exogenous gene operatively linked to a promoter that is upregulated by a transactivator protein; b) providing a second transgenic parent animal whose genome comprises i) a second transgene, comprising a second promoter that is downregulated by the transactivator protein and operatively linked to an antisense gene that encodes a sequence which inhibits or reduces processing of the F transgene transcript; and ii) a third transgene comprising a tissue specific promoter operatively linked to a gene encoding the transactivator protein; and c) breeding the first transgenic parent animal with the second transgenic parent animal to provide a transgenic offspring animal whose genome comprises the three transgenes. The present invention also relates to a DNA construct comprising the second transgene and to the second transgenic parent mouse used in the method.

### BACKGROUND

Controllable, tissue specific expression of transgenes is a primary goal in transgenic animals that serve as experimental models of disease. Early attempts at controlling transgene expression in transgenic mice have often employed transgenes that had been operably linked to tetracycline-responsive promoters or to rapamycin-responsive promoters.

The first tetracycline-controlled promoter was constructed by fusing the operator sequence of the E. coli tetracycline-resistance gene (tetO) to the minimal promoter sequence of the human cytomegalovirus immediate-early gene (hCMV IE). (Gossen, M. and Bujard, H., Tight control of gene expression in mammalian cells by tetracycline-responsive promoters. Proc.NatLAcadSci. USA 89(12), 5547-5551, 1992.) As shown in FIG. 1, this promoter (tetO/hCMV) is activated when it binds to a fusion protein constructed from the tetracycline-controlled transactivator (tTA), which contains a tetO binding protein tetracycline repressor (tetR), plus a transcription activator, virion protein 16 (VP 16), from herpes simplex virus. In the absence of tetracycline, this fusion protein binds to the tetO/hCMV promoter and activates transcription of the exogene. In the presence of tetracycline, exogene transcription is blocked because tetracycline binds to the transactivator (tTA) and interferes with its binding to the tetO/hCMV promoter. Since tetracycline down-regulates transgene expression, this is called the “tet-Off” promoter system.

Unfortunately, the tet-Off promoter system is associated with leaky gene expression which complicates the use of this system for basic research or pharmaceutical application. Furth and colleagues first used the tet-Off tetracycline-controlled promoter in transgenic mice that expressed the reporter transgene, luciferase, under control of the tetO/hCMV promoter. When tetracycline was absent, luciferase expression was observed in numerous tissues. When tetracycline was provided subcutaneously, the luciferase activity was significantly reduced to low, but still detectable, background levels. This leaky transgene expression was observed with two different plasmid delivery systems, the paired plasmid system of Furth (Furth, P. A., St.Onge, L., Boger, H., Gruss, P., Gossen, M., Kistner, A., Bujard, H., and Henninghausen, L., Temporal control of gene expression in transgenic mice by tetracycline responsive promoter. Proc.Natl. Acad Sci. USA 91(20), 9302-9306, 199) and the combined system of Nathalis (Schultze, N., Burki, Y., Lang, Y., Certa, U., and Bluethmann, H., Efficient control of gene expression by single step integration of the tetracycline system in transgenic mice. Nature Biotechnology 14(4), 499-503, 1996.).

Similarly, leaky transgene expression has also been observed in transgenic models that utilized the tet-Off system linked to a tissue specific promoter, the cardiac-specific, α-myosin heavy chain promoter (α-mhc) (Yu, Z., Redfern, C. S., arid Fishman, G. I., Conditional transgene expression in the heart. Circ.Res. 79(4), 691-697, 1998). In these studies, the leaky transgene expression was observed in various tissues, such as kidney, skeletal muscle, pancreas, and live. Even stronger leaky gene expression was observed in cardiac tissues.

The tetO/hCMV genetic system has also been modified to allow tetracycline to induce, rather then inhibit, transgene expression. As shown in FIG. 2, the modified system employs the reverse tetracycline-controlled transactivator (rtTA), comprised of a mutated tetO binding protein, rtetR (tetracycline repressor) linked to VP16 (Gossen, M., Freundlieb, S., Bender, G., Muller, G., Hillen, W., and Bujard, H., Transcriptional activation by tetracyclines in mammalian cells. Science 268(5218), 1766-1769, 1995). When tetracycline is absent, the rtTA cannot bind to the tetO in the tetracycline-controlled promoter. When tetracycline is present, it binds to the rtTA which allows the rtTA to bind to the tetO in the promoter and up-regulate transcription of the exogene. Since tetracycline induces gene expression, this is called the “tet-On” promoter system.

Leaky gene expression is less prevalent in transgenic mice in which the transgene is under control of the tet-On promoter as compared to the mice in which the transgene is under control of the tet-Off promoter. Nonetheless, use of the tet-On promoter is not able to eliminate residual leaky gene expression due to the limited CMV promoter. In addition, transgenic mice whose transgenes are responsive to rapamycin have also been shown to express detectable background levels of transgene transcription in the absence of rapamycin. Such “leaky” transgene transcription seriously compromises studies with these transgenic mice.

Accordingly, it is desirable to have new methods and systems for producing transgenic animals, particularly transgenic mice, which have little to no background transgene transcription, particularly in specific tissues.

### SUMMARY OF THE INVENTION

The present invention provides a method and system for controlling the expression of transgene products in specific tissues in a transgenic animal, particularly a transgenic mouse, while eliminating background expression of the transgene products. The method comprises:

a) providing a transgenic parent animal referred to hereinafter as a “P2” animal, whose genome comprises a transgene, hereinafter the “F” transgene, comprising an exogenous gene operatively linked to a promoter, hereinafter the “F” promoter; wherein the F promoter is upregulated by a transactivator protein; and wherein interaction of the transactivator protein with the F promoter occurs in the presence of a transactivator regulator;

b) providing another transgenic parent animal, referred to hereinafter as a “P1” animal, whose genome comprises

i) a second transgene, hereinafter the “S” transgene, comprising a second promoter, hereinafter the “S” promoter, operatively linked to an antisense gene; wherein the S promoter is downregulated by the transactivator protein; wherein interaction of the transactivator protein with the S promoter occurs in the presence of the transactivator regulator; and wherein the antisense gene encodes a sequence which inhibits or reduces processing of the F transgene transcript; and

ii) a third transgene, hereinafter the “T” transgene, comprising a tissue specific promoter operatively linked to a gene encoding the transactivator protein; and

c) breeding the transgenic parent animal of step (a) with the transgenic parent animal of step (b) (e.g., a P1 mouse with a P2 mouse), to provide a transgenic offspring animal, hereinafter the “F1” animal, whose genome comprises the F transgene, the S transgene and the T transgene.

As shown in FIG. 3, administration of a transactivator regulator, such as for example tetracycline, to an F1 mouse produced in accordance with the above described method, results in enhanced expression of the exogenous gene and reduced expression of the antisense gene product in target tissues. As compared to a P2 mouse, the F1 mouse has reduced levels of the exogenous gene product in the absence of the transactivator regulator, The F1 mouse exhibits tissue specific expression of the exogenous gene product following administration of the transactivator regulator and little to no background expression of the exogenous gene product in the absence of the transactivator regulator. Accordingly, the F1 mouse is a research tool useful for studying the impact of the transgene product on the biology of the mouse under defined conditions of expression.

The system for controlling expression of a desired transgene product in specific tissues of a transgenic mouse while eliminating background expression of the desired transgene product comprises a P1 animal, preferably a P1 mouse, and a vector or a DNA construct for producing the P2 animal, which is also preferably a mouse. In a preferred embodiment of the P1 animal, the S transgene comprises a CMV promoter, a tetracycline operator sequence, and a sequence encoding an antisense RNA which binds to the 5′ untranslated sequence (5′ UTR), and more preferably the first intron, of the human CMV immediate-early gene; and the T transgene comprises a tissue specific promoter operatively linked to a reverse tetracycline transactivator gene, hereinafter the “rtTA” gene. In the preferred embodiment, the vector or DNA construct for producing the P2 animal comprises a tetracycline controlled promoter, hereinafter the “tetO/hCMV” promoter, and multiple restriction sites for subcloning a desired exogene. The tetO/hCMV promoter, comprises the operator sequence of the E. coli tetracycline-resistance gene fused to the minimal promoter sequence of the human cytomegalovirus immediate-early gene. In another embodiment, the vector or DNA construct further comprises the desired exogene operatively linked to the tetO/hCMV promoter.

The present invention also relates to a DNA construct comprising the S transgene. The antisense gene of the S transgene comprises a sequence which binds to the F transgene transcript to inhibit or reduce translation of the F transgene transcript or splicing of the F transgene transcript. The DNA construct is useful for preparing the P1 animal.

The present invention also relates to a P1 mouse. In a preferred embodiment, the P1 mouse comprises a S transgene comprising CMV promoter, a tetracycline operator sequence, and a sequence encoding an antisense RNA which binds to the 5′ untranslated sequence and the intron of the human CMV immediate-early gene; and a T transgene comprising a tissue specific promoter operatively linked to the rtTA gene.

### BRIEF DESCRIPTION OF THE FIGURES

FIG. 1 depicts the standard Tet-Off gene expression system.

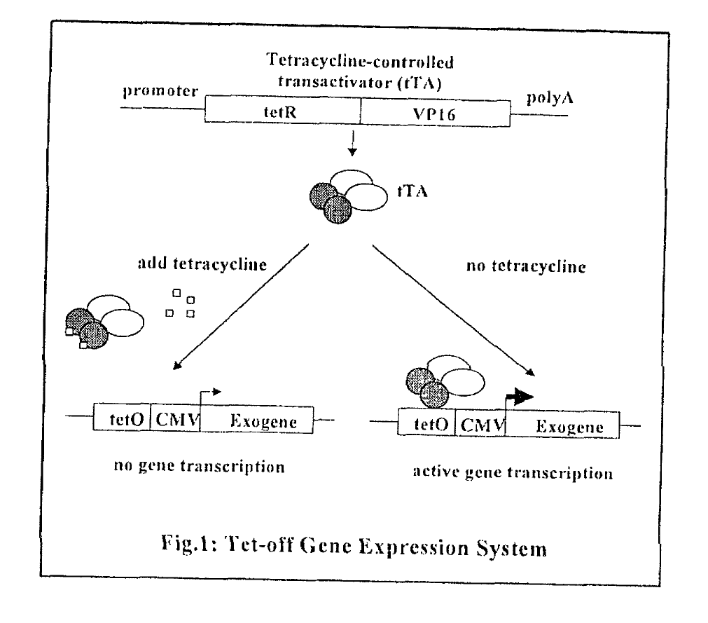

FIG. 2 depicts the standard Tet-On gene expression system.

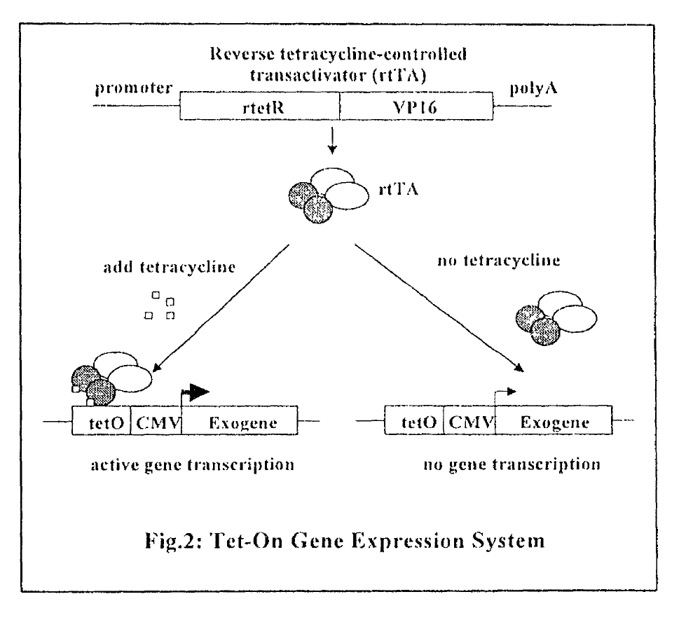

FIG. 3 depicts a tetracycline double controlled tissue specific gene expression system

FIG. 4 depicts the tetracycline-controlled hCMV antisense gene expression system

FIG. 5 depicts the design of the gene expression plasmids described in the Examples.

FIG. 6 is a schematic showing construction of pVRBtA.

FIG. 7 is a schematic showing construction of pTo 1012.

FIG. 8 is a schematic showing construction of pToLd.

FIG. 9 is a schematic showing construction of pTo1412 and pToluci plasmids

FIG. 10 is a schematic showing construction of Mhc-tetON plasmid.

FIG. 11 is a map of pClone 22.

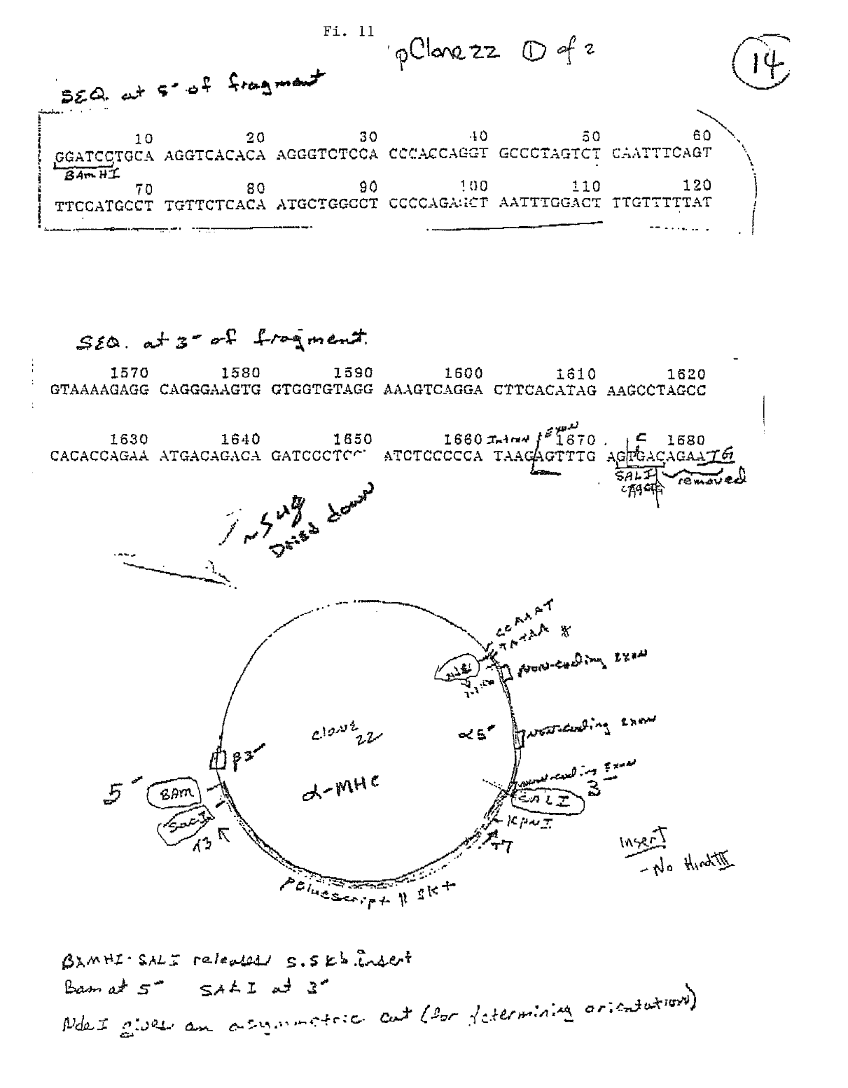

FIG. 12 is a map of pUHD10-3. The plasmid consists out of three main fragments: pBR322-sequences including colE1-origin of replication, β-lactamase-resistance-gene with the Pbla/p3 of Tn2661 (HincII-site and PstI-site removed); the regulatory region with hCMV minimal promoter (−53 relative to start site) with heptomerized upstream tet-operators as described in PNAS 89, 5547-51 (1992); multiple cloning site, MCS, containing SacII, EcoRI and XbaI recognition sequences; and SV40 polyadenylation downstream of the MCS.

FIG. 13 is a map of pL^{d}.444.

FIG. 14 is a map of ICAM2/Ul/tTA

FIG. 15 depicts the tetracycline triple controlled transgenic system.

FIG. 16 is a schematic showing construction of pToGFP and pToRFP plasmids.

### DETAILED DESCRIPTION OF THE INVENTION

The present invention provides a method for controlling the expression of transgene products in specific tissues in a transgenic animal while eliminating background expression of the transgene products in tissues of the animal. The method comprises providing a P1 animal and a P2 animal and breeding the P1 animal with the P2 animal to provide an F1 animal. The P1, P2, and F1 animals are non-human mammals such as, for example, pigs. Preferably, the P1 animal, the P2 animal, and the F1 animal are mice.

The P2 animal is a transgenic parent animal whose genome comprises an F transgene which comprises an exogenous gene operatively linked to an F promoter that is upregulated by a transactivator protein. Interaction of the transactivator protein with the F promoter occurs in the presence of a transactivator regulator. Advantageously, the P2 animal can comprise any desired exogenous gene. As used herein an exogenous gene is a gene that is not normally present in the genome of the P2 animal. The exogenous gene can be a reporter gene such as for example a gene which encodes luciferase or beta-galactosidase, green fluorescence protein (GFP) or red fluorescence protein (RFP). Clones comprising such reporter genes are commercially available. Alternatively, the exogenous gene encodes a biologically active molecule. An example of such gene is the H-2L^{d} gene which encodes the H-2L^{d} protein. Preferably, the P2 animal is homozygous for the F transgene.

In a preferred embodiment the F promoter is a tetracycline controlled promoter. The tetracycline controlled promoter comprises the operator sequence of the E. colitetracycline-resistance gene fused to the minimal promoter sequence of the human cytomegalovirus immediate-early gene. Preferably, the tetracycline controlled promoter further comprises the first intron of the human CMV immediate early gene. Preferably, the P2 animal further comprises a transgene comprising a constitutive promoter operatively linked to an antisense gene which encodes a transcript that blocks processing of the exogene transcript.

The genome of the P1 animal comprises an S transgene comprising an S promoter operatively linked to an antisense gene which encodes a transcript that blocks processing of the exogene transcript, i.e., the F transgene transcript, and a T transgene comprising a tissue specific promoter operatively linked to a gene encoding the transactivator protein. The S promoter is downregulated by the transactivator protein which up-regulates the S promoter of the S transgene. Interaction of the transactivator with the S promoter occurs in the presence of the transactivator regulator. The S antisense gene encodes a sequence which blocks the mRNA splicing of the F transgene transcript. The P1 animal, expresses the transactivator protein in target tissues, i.e., those tissue corresponding to the tissue specific promoter. In the absence of the transactivator regulator the P1 animal also expresses the antisense gene product. Administration of the transactivator regulator to the P1 animal results in reduced expression of the antisense gene product. In a preferred embodiment, the transactivator regulator is tetracycline and the transactivator protein is the rtTA protein.

The genome of the F1 animal comprises the F transgene, the S transgene, and the T transgene. In the absence of the transactivator regulator, the F1 animal expresses the antisense gene product in all tissues and the transactivator protein in target tissues. Administration of the transactivator regulator to the F1 animal results in enhanced expression of the exogenous gene and reduced expression of the antisense gene product in target tissues. The F1 animal, particularly an F1 mouse, is a research tool useful for studying the impact of the transgene product function on the biology of the animal under defined conditions of expression. As compared to the P2 animal, the F1 animal has reduced levels of the exogenous gene product in the absence of the transactivator regulator, Accordingly, the F1 animal exhibits tissue specific expression of the exogenous gene product following administration of the transactivator regulator and little to no background expression of the exogenous gene product in the absence of the transactivator regulator.

The system for controlling expression of a desired exogene product in specific tissues of a transgenic mouse while eliminating background expression of the desired exogene product comprises the P1 animal, which is preferably a P1 mouse, and a vector or a DNA construct for producing the P2 animal, which, preferably, is also a mouse. In a preferred embodiment of the P1 animal, the S transgene comprises a CMV promoter, a tetracycline operator sequence, and a sequence encoding an antisense RNA which binds to the 5′ untranslated sequence (5′ UTR), and more preferably, the first intron of the human CMV immediate-early gene,; and the T transgene comprises a tissue specific promoter operatively linked to a reverse tetracycline transactivator gene, i.e., the “rtTA” gene. Any tissue specific promoter is suitable for use in the present invention. Thus, if one desires to study the impact of the exogene product in endothelial tissues, then the tissue specific promoter is selected to be an endothelial cell specific promoter, such as for example an ICAM-2 promoter. Similarly, if one desires to study the impact of the exogene product in cardiac myocytes, then the tissue specific promoter is selected to be a cardiac myocyte specific promoter, such as for example the alpha-myosin heavy chain promoter. The present system can easily be modified to accommodate different exogenes and different tissue specific promoters.

In the preferred embodiment, the vector or DNA construct for producing the P2 animal comprises a tetracycline controlled promoter and multiple restriction sites for subcloning a desired exogene. The tetracycline controlled promoter or tetO/hCMV promoter comprises the operator sequence of the E. coli tetracycline-resistance gene fused to the minimal promoter sequence of the human cytomegalovirus immediate-early gene. Preferably, the construct comprises both the 5′ UTR and the first intron of the human cytomegalovirus (CMV) immediate early gene. In an alternative embodiment, the vector or DNA construct further comprises an exogenous gene such as for example a reporter gene or a coding sequence for a biologically active molecule. The preferred system allows for tetracycline controlled, tissue specific expression of any exogene, and eliminates unwanted background expression of the product of such exogene.

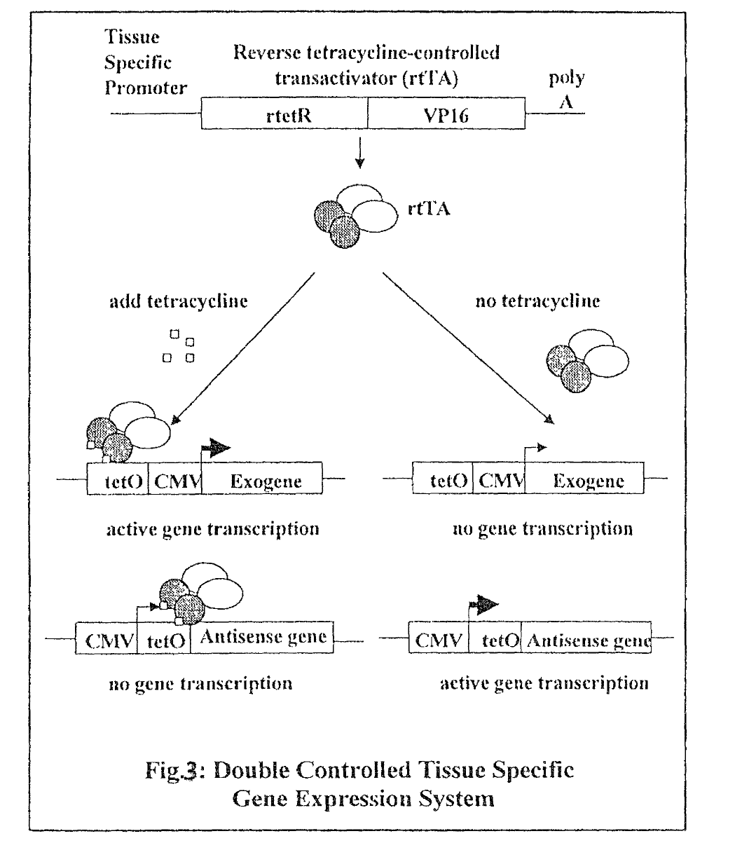

The present invention also provides a DNA construct comprising the S transgene. In a preferred embodiment, the construct comprises a sequence encoding an antisense RNA which binds to a sequence selected from the group consisting of the 5′ untranslated sequence human CMV immediate-early gene, the first intron of human CMV immediate early gene, or a combination thereof. The preferred S transgene further comprises a CMV promoter, and a tetracycline operator sequence operably linked to the antisense RNA coding sequence. As shown in FIG. 3, the tetracycline operator sequence is located at the 3′ end of the CMV promoter and the 5′ end of the antisense RNA encoding sequence. The DNA construct is useful for preparing the P1 animal.

The present invention also relates to a P1 mouse. In a preferred embodiment, the P1 mouse comprises a S transgene comprising a sequence encoding an antisense RNA which binds to the 5′ untranslated sequence and the intron of the human CMV immediate-early gene, a CMV promoter, and a tetracycline operator sequence; and a T transgene comprising a tissue specific promoter operatively linked to the rtTA gene. Preferably, the P1 mouse is homozygous for the S transgene and the T transgene.

### EXAMPLES

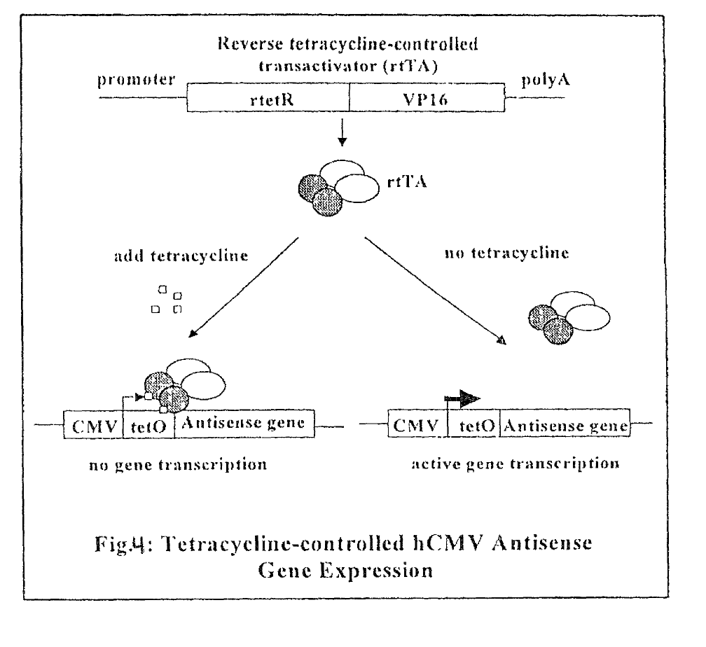

The following examples are for purposes of illustration only and are not intended to limit the scope of the invention as defined in the claims which are appended hereto. The design of the gene expression plasmids used to prepare the P1 and P2 animals described in the following examples is shown in FIG. 4. The references cited in this document are specifically incorporated herein by reference.

### Example 1

### Tetracyline-upregulatable Vectors

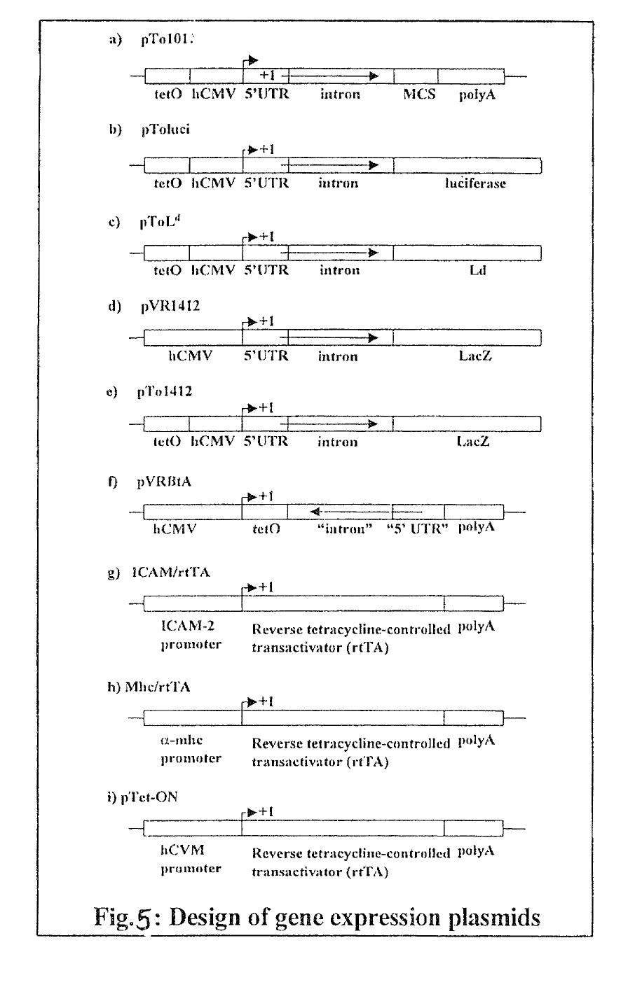

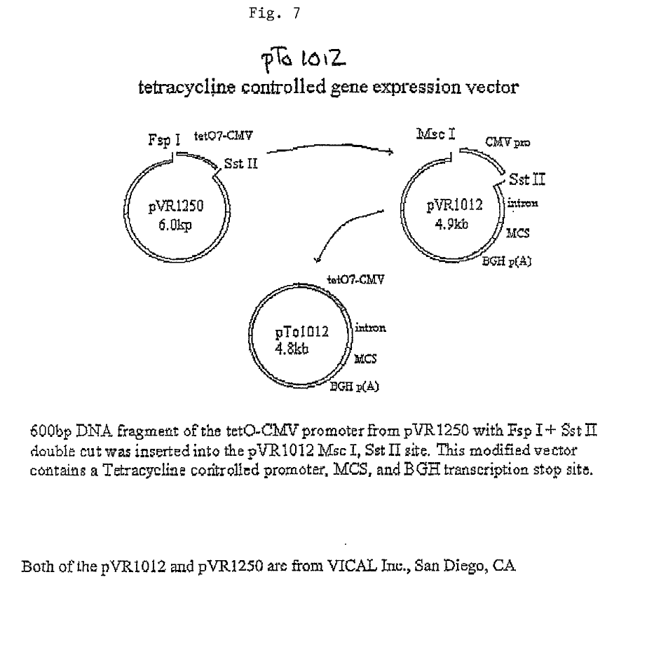

A. The tetracycline-controlled promoter (tetO/hMHC) was obtained from the plasmid pVR1250 (Vical, Inc. San Diego, Calif.) with Fsp I and Sst II double cut and replaced the hCMV promoter of the high expression vector pVR1012 (Vical Inc.). This modified vector, pTo1012, contains the tetracycline-controlled promoter, multiple subclone sites, and the Bovine Growth Hormone transcription terminated sequence (FIG. 5a and FIG. 7). The pTo1012 plasmid can be used to build any exogene expression construct.

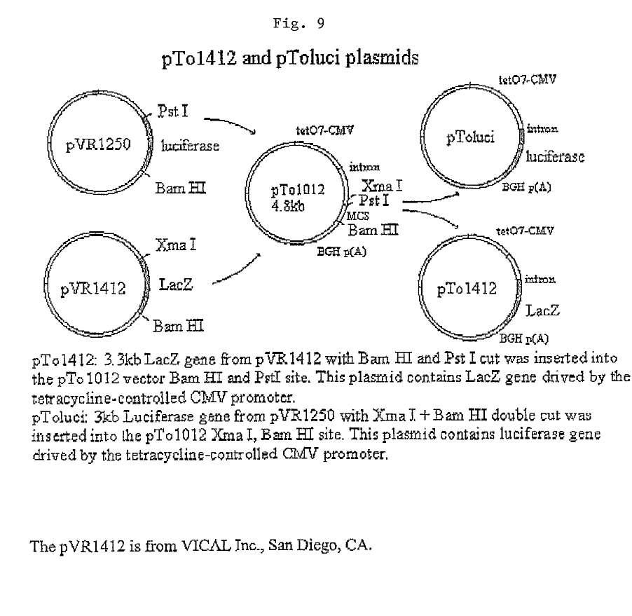

Vector function was tested in vitro by inserting a luciferase gene (pToLuci, FIG. 5b and FIG. 9), followed by transfection into COS 1 cells (Cowan, P. J., Shinkel, T. A., Witort, W. J., Barlow, H., Pearse, M. J., and d'Apice, A. J. F., Targeting gene expression to endothelial cells in transgenic mice using the human intercellular adhesion molecule 2 promoter. Transplantation 62(2), 155-160, 1996; and Subramaniam, A., Jones, W. K., Gulick, J., Wert, S., Neumann, J., and Robbins, J., Tissue specific regulation of the alpha-myosin heavy chain gene promoter in transgenic mice. J.Biol.Chem 266(36), 24613-24620, 1991.). The results demonstrated that luciferase gene expression in the pToluci transfected COS 1 cells can be up regulated by tetracycline.

B. Tetracycline-controlled H-2Ld Expression Plasmid.

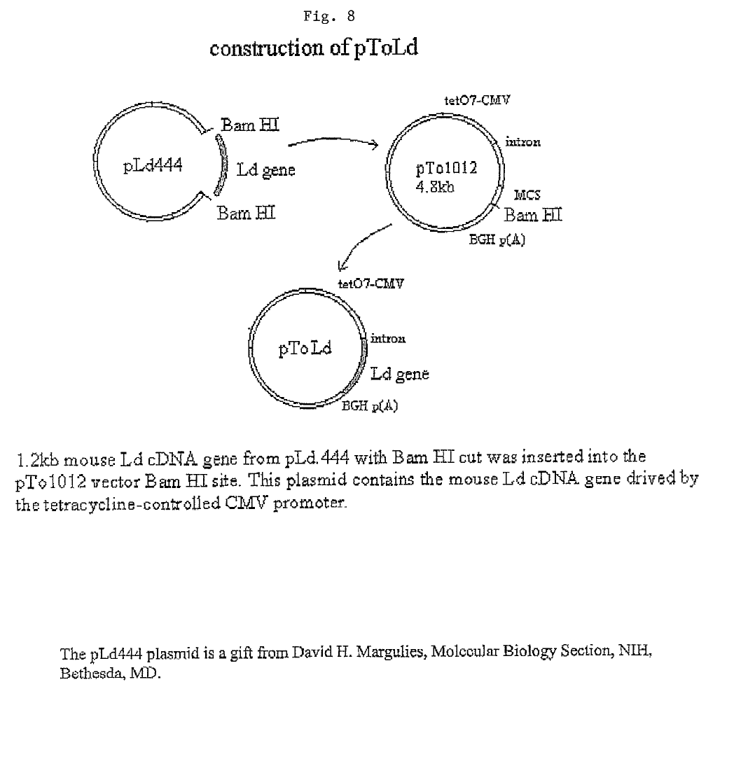

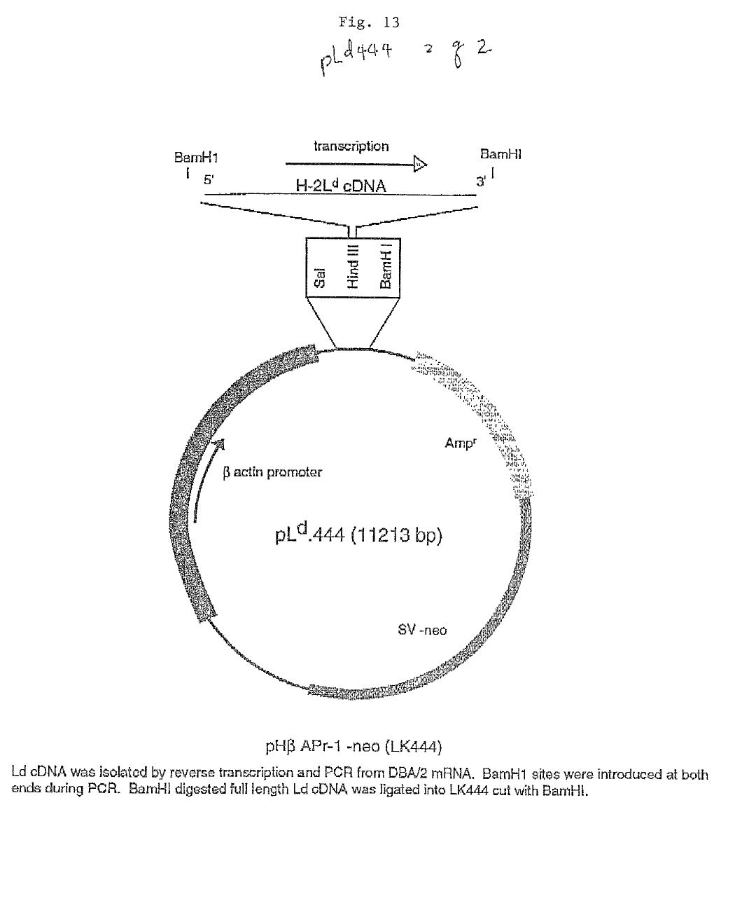

The mouse H-2Ld cDNA from the pLd.444 plasmid (see FIG. 13) was digested with Bam HI and inserted into the Bam HI site of the pTo1012 subclone site. The new plasmid pToLd (FIGS. 5e and 8) contains the murine H-2Ld gene driven by the tetracycline-controlled hCMV promoter.

C. Tetracycline-controlled β-galactosidase Expression Plasmid

The β-galactosidase gene from the pVR1412 plasmid (FIGS. 5d and 9), a gift from Vical Inc.) was double digested with Bam HI and Pst I. The 3.3 kb DNA fragment was inserted into the pTo1012 vector. The pTo1412 plasmid (FIGS. 5e and 9) contains the β-galactosidase gene driven by the tetracycline-controlled promoter. Function of the pTo1412 was tested by co-transfection of COS 1 cells with this plasmid plus the pTet-On plasmid (Clonetech Laboratories, Inc. Palo Alto, Calif.), which encodes the reverse tetracycline-controlled transactivator. The results showed that the β-galactosidase gene in the pTo1412 transfected COS1 cells can be up-regulated by tetracycline.

### Example 2

### Tetracycline Down-regulated Antisense Gene Expression Construct

A. Preparation of the Construct

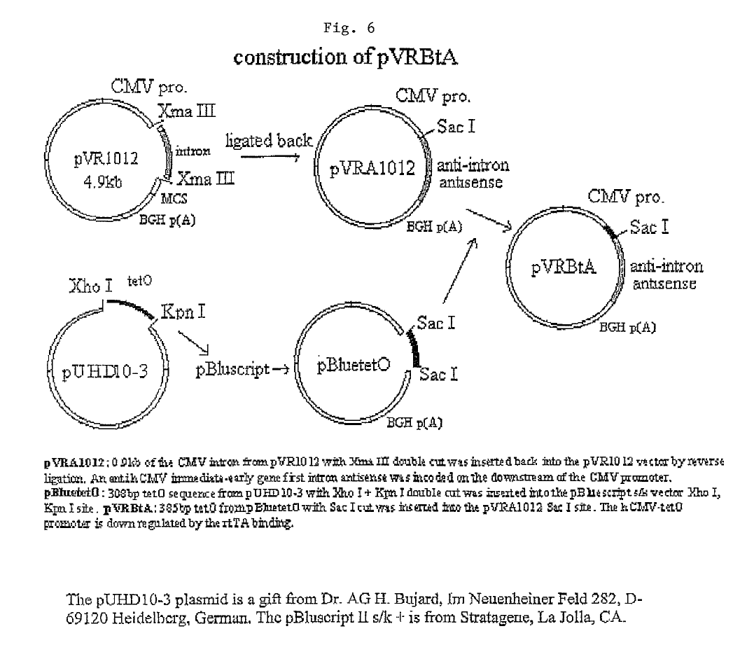

A tetracycline-controlled antisense expression plasmid, pVRBtA was generated as shown in FIG. 6. The plasmid pVRBtA expresses a 0.9 kb antisense RNA which is highly specific, and binds only to the 5′ untranslated sequence and the intron of the hCMV immediate-early gene. This antisense blocks the splicing of any mRNA which is transcripted from the hCMV promoter. To prepare the plasmid, an antisense expression plasmid was constructed by reverse ligation of the 0.9 kb Xma III fragment which contains the 5′ UTR and the intron of the hCMV IE1 gene back into a pVR1012 vector. Next, the CMV promoter was modified into a tetracycline-controlled promoter by inserting a tetracycline operator (tetO) sequence at the Sac I site that is right at the 3′ end of the CMV promoter (FIG. 4f). When the transactivator (rtTA) binds to the tetO site, the mRNA transcription from the CMV promoter is blocked by the space effect. Therefore, the expression of the antisense from the pVRBtA is down regulated by the presence of tetracycline. (See FIG. 3.)

B. Antisense RNA Detection

COS1 cells were transfected with 1.5 μg of pVRBtA using LipofectAMINE (Life Technologies, BRL, Gaithersburg, Md.). After 48 hr culturing, the total RNA was isolated with the neutral-phenol extraction method. Plasmid DNA contamination was removed by DNase digestion at 37° C. for 2 hours. The total cDNA was obtained by reverse-transcription. The primer pairs for the antisense RNA detection (sense: 5′-AGA AGA CAC CGG GAC CGA TC-3′, and antisense: 5′-ATG TAA TCG CAG CCG TCG TAG-3′) were designed to produce a DNA fragment of 243 bp. For PCR, 30 cycles were used (94° C./30 sec, 60° C./20 sec, and 72° C./1 min.). The results showed that the antisense RNA was detectable in the transfected COS cells via RT-PCR.

C. Antisense Function Assay

The blocking function of the antisense construct was tested by co-transfection of COS 1 cells with the luciferase reporter plasmid, pToLuci, and either the hCMV antisense plasmid, pVRBtA or the control plasmid, pVR1012. The in vitro blocking results showed that the antisense plasmid is able to block 40 to 45% activity of the reporter gene. This is significant blocking by antisense, considering that the inhibition in this system can be observed only when the COS 1 cells are co-transfected.

### Example 3

### Tissue Specific Expression Systems

A Endothelial Specific Expression System.

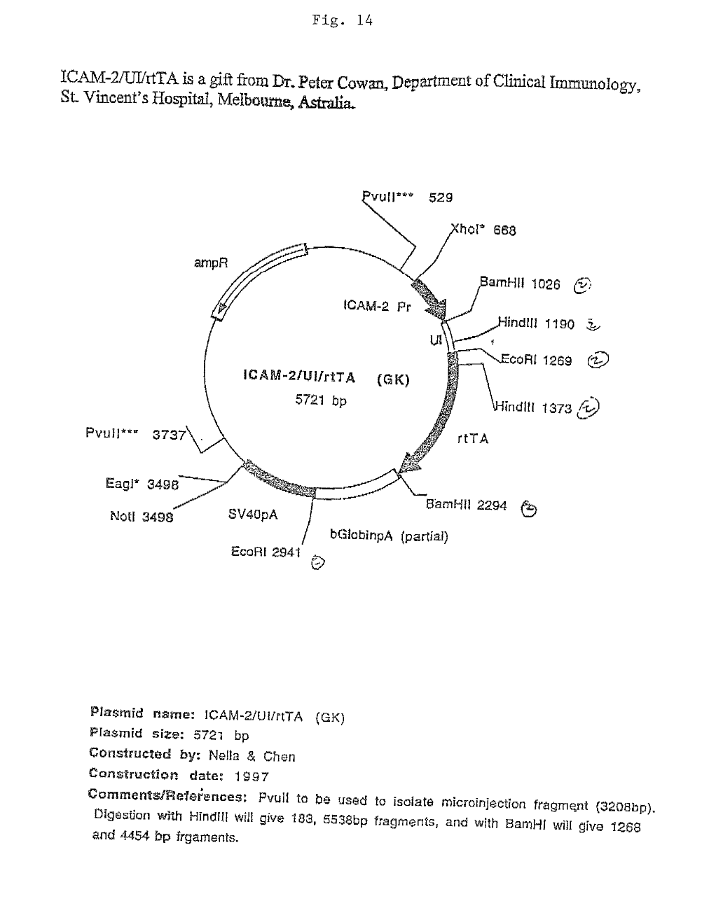

The ICAM-2 promoter is a tissue specific transcription promoter which drives gene expression only in endothelial cells (Volloch, V., Schweitzer, B., and Rits, S., Inhibition of pre-mRNA splicing by antisense RNA in vitro: effect of RNA containing sequences complementary to introns. Biochem Biophys Res Commun 179(3), 1600-1605, 1991). The hCMV promoter in the pTet-On plasmid was replaced with the ICAM-2promoter (obtained from Dr. Peter Cowan at the St. Vincent's Hospital). This plasmid construct, pICAM/rtTA (FIGS. 5g and 14), contains the reverse tetracycline-controlled transactivator (rtTA) under ICAM-2 promoter control.

This endothelial expression system was tested in MAE cells, a murine endothelial cell line derived from aortic endothelial cells (obtained from Dr. Robert Auerbach at the University of Wis.). The MAE cells were co-transfected with the pICAM/rtTA plasmid and pVR1250 luciferase reporter plasmid, which contains a luciferase gene driven by a tetracycline-controlled promoter. When tetracycline was present at a concentration of 5 μg/ml, luciferase activity was up-regulated. This regulated system was not operative in COS1 cells, which are not of endothelial origin. These results indicate that the pICAM/rtTA plasmid is able to express the rtTA transactivator, and that this expression is tissue specific.

B. Cardiac Myocyte Tissue Specific Expression System.

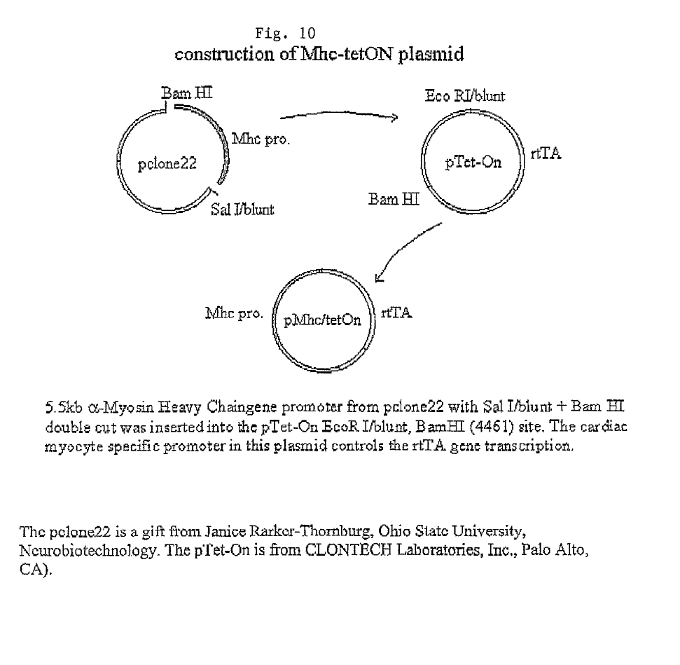

The cardiac myocytes specific promoter, a-myosin heavy chain promoter (Mhc), is another promoter that can be used for generating tissue specific expression systems. The hCMV promoter in the Tet-On plasmid was removed (Sal I/blunted and the Barn HI double cutting) and replaced by the α-myosin heavy chain promoter, 5.5 kb Eco RI (blunt) and Bam HI fragment (FIGS. 5h and 10).

### Example 4

### ICAM-2-tet-On/hCMV Antisense Transgenic Mice (PIE Line)

The PIE line, a transgenic mouse line that displays endothelial-specific rtTA expression, was generated by microinjection of C57BL/6 embryo with the I-CAM-1-rtTA and the pVRBtA plasmids.

The pICAM-1-rtTA plasmid contains the rtTA gene driven by the endothelial specific ICAM-2 promoter in a cassette that can be isolated by digestion with Pvu II restriction endonuclease. The plasmid, pVRBtA, contains the hCMV antisense gene driven by the tetracycline down regulated promoter in a cassette that can be isolated with Xmn I/Msc I restriction endonucleases. These two gene expression cassettes were isolated and purified by gel electrophoreses. Three pico-liters of these two purified DNA constructs, in a concentration of 3 ng/μl, were co-injected into the C57BL/6 embryo by micro-injection. In theory, these PIE mice should express the genes of the reverse tetracycline-controlled transactivator (rtTA) protein only in endothelial cells, whereas they should expression the hCMV-antisense gene in multiple tissues (in the absence of tetracycline).

A. Gene Screening

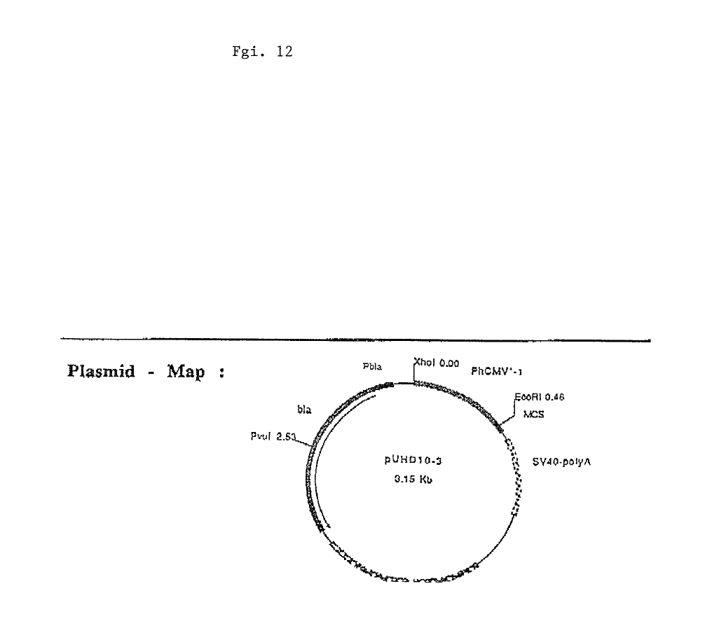

Putative P1E transgenic mice were screened by PCR for detection of rtTA gene and the antisense gene sequence. Tail tissues (3 mm) from transgenic mice were digested with 25 μl 0.1% collagenase at 37° C. for 2 hr. After a phenol and chloroform extraction to obtain DNA, 5 μl of DNA was analyzed by PCR. The PCR primer pair for rtTA transgene detection (sense: rtTA-5, 5′-AGA TCA AGA GCA TCA AGT CG-3′ and antisense: rtTA-3, 5′-AGT CGG CCA TAT CCA GAG-3′) was designed to produce a DNA fragment of 512 bp (FIG. 18a). PCR used 30 cycles for analysis with these primer pairs, (94° C./60 sec, 57° C./60 sec, and 72° C./60/cycle). The primer pair for hCMV antisense gene detection (sense: 5′-AGA AGA CAC CGG GAC CGA TC-3′ and antisense: S′-GAA GAA GAT GCA GGC AGC TGA G-3′) was designed to produce a DNA fragment of 243 bp (FIG. 12c), which is not present in the normal B6 genome. For PCR analyses, 30 cycles was used (94° C./60 sec, 61° C./60 sec, and 72° C./60 sec/cycle).

B. Detection of the rtTA and laCMV Antisense Gene Products in PIE Transgenic Mice.

The rtTA gene product was detected by Western blot with a specific anti-Tet monoclonal antibody (CLONTECH Laboratories, Inc.). Since the rtTA gene is controlled by the ICAM-2 promoter, the rtTA mRNA and protein is transcribed and translated in endothelial cells. Since tail and pinnae are endothelial cell-rich tissues, either may b used for detection of the rtTA. Tissues from PIE transgenic mice, found to be DNA-positive by gene screening for both rtTA and hCMV antisense, were homogenized and treated with SDS PAGE sample buffer at 100° C. for 5 minutes. The total proteins were separated by PAGE and blotted into a PVDF membrane. A 37 kDa of rtTA protein is specifically detected with the mouse anti-TetR monoclonal antibody, which binds to the TetR domain in the tTA or rtTA fusion proteins (CLONTECH Laboratories, Inc., Palo Alto, Calif.).

The hCMV antisense RNA product is detected by the RT-PCR technique. Since it is expected that the CMV promoter for the hCMV antisense gene will be expressed in multiple tissues, tail or pinna tissues can also be used for the screening of the hCMV antisense RNA transcription. Total RNA from the tissues of the P1E transgenic mice found to be positive in the DNA screening for rtTA and hCMV antisense, is isolated by the neutral-phenol extraction method. The contaminating DNA is removed by DNase digestion at 37° C. for 2 hr. Then the RNA is reverse transcribed into cDNA with the reverse-transcriptase. The cDNA is tested by PCR with the same primers used above for the antisense RNA detection.

c. Selection of Tissue Specific rtTA Expression Mouse Lines.

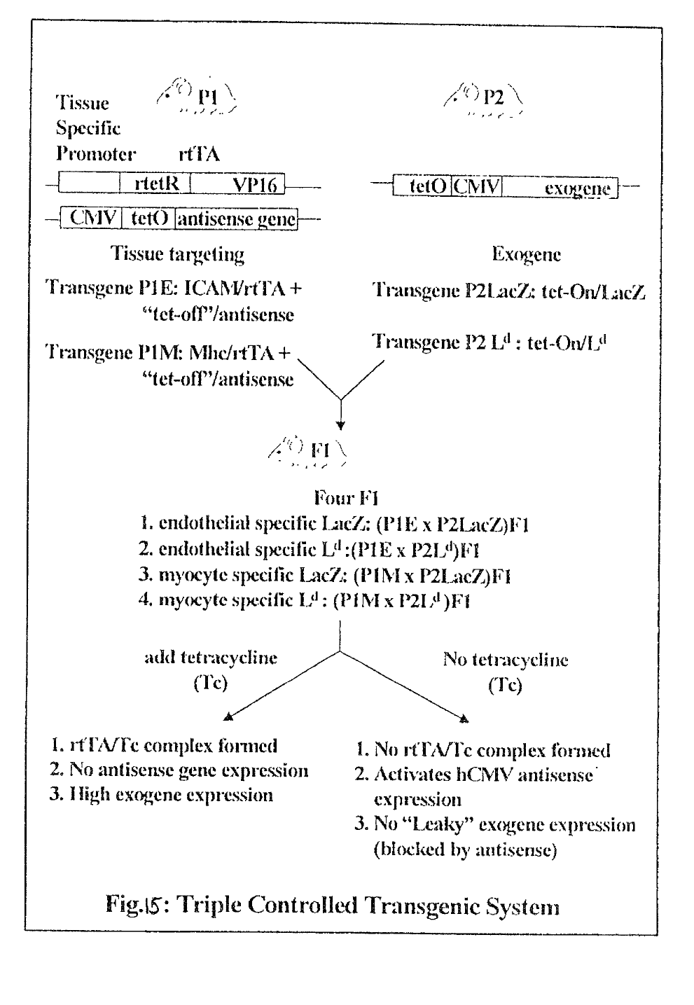

Endothelial specific rtTA expression in the PIE mice is evaluated by immunohistochemistry. Ear tissues from the PIE transgenic mice (rtTA gene expression positive in Western blot and antisense RNA positive in RT-PCR) is embedded with Tissue Tek OCT (Miles, inc., Elkhart, Ind.) then sectioned in 5 μm. The biotin-labeled mouse anti-TetR mAb and streptavidin-Horseradish Peroxidase is used for the rtTA protein staining. Additional tissues, such as liver, heart, kidney, muscle, and spleen are screened for endothelial-specific rtTA expression. The PIE transgenic mice with endothelial specific rtTA expression are used to prepare endothelial specific F1 mice as shown in FIG. 15

### Example 5

### Alpha-Myosin-tet-On/hCMV Antisense Transgenic Mice (PIM)

The P1 M line, a transgenic mouse line that displays myocyte-specific rtTA expression, was generated by use of the pMhc/rtTA and the pVRBtA plasmids. As shown in FIG. 10, the plasmid, MHC/rtTA contains the rtTA gene driven by the cardiac myocyte specific alpha-Myosin Heavy Chain promoter in a cassette that can be isolated by digestion with Xho I/Hind III restriction endonucleases. The antisense plasmid, pVRBtA, was digested with Xmn I/Msc I restriction endonucleases as before. These two gene expression cassettes were isolated, and purified by gel electrophoreses. Three pico-liter of these two purified DNA constructs, in a concentration of 3 ng/μl, were co-injected into the C57BL/6 embryo by microinjection. In theory, these PIM mice should express the genes for the reverse tetracycline-controlled constitutive rtTA protein only in cardiac myocyte, whereas the hCMV antisense RNA gene should be expressed in multiple tissues.

a. Gene Screening.

The identical methods described for the screening of the PIE mice as described in Example 4 are used for the P1M mice.

b. Detection of the rtTA and hCMV Antisense Gene Products in PIM Transgenic Mice.

The identical methods described for gene product detection in the PIE mice as described in Example 4 are used to test heart tissues from the PIM mice.

c. Selection of a Tissue Specific rtTA Expression Mouse Lines.

Cardiac myocyte-specific rtTA expression in the PIM mice is evaluated by immunohistochemistry. Heart, liver, kidney, skeletal muscle, and spleen tissues from the PIM transgenic mice, found to be positive for rtTA gene product and hCMV antisense RNA, is embedded with OCT and sectioned. The biotin-labeled mouse anti-TetR mAb and streptavidin-HRP is used for the rtTA protein staining. The rtTA should be detected only in myocytes, but not endothelial cells of the cardiac tissues. None of the other tissues should express rtTA. The littermates of these rtTA positive PIM transgenic mice are used to prepare F1 mice expressing myocyte specific exogenes as shown in FIG. 15.

### Example 6

### Tetracycline-controlled H-2 Ld Transgenic Mice (P2Ld)

The P2Ld line, a transgenic mouse line that displays tetracycline transactivator-controlled H2Ld expression in all tissues, is generated with the plasmid, pToLd, which contains the mouse H-2Ld gene driven by the tetracycline-controlled promoter in a cassette that can be isolated by digestion with Xmn III/Xho I restriction endonucleases. This gene expression cassette will be isolated., and purified by gel electrophoreses. Three pico-liter of these two purified DNA constructs, in a concentration of 3 ng/μl, are co-injected into the C57BL/6 embryo by microinjection with the identical technique as described in Example 4. The P2Ld mice, should not express H-2L^{d} protein in any tissue without the tetracycline-controlled transactivator and tetracycline present. However, the background or leaky expression of the H-2L^{d} gene from the hCMV minimal promoter should be found in multiple tissues.

a. Gene Screening.

Putative P2Ld transgenic mice are screened by PCR analysis of DNA from tail tissues using the primer pair (sense: 5′-AGA AGA CAC CGG GAC CGA TC-3′ and antisense: 5′-ATG TAA TCG CAG CCG TCG TAG-3′). This primer pair specifically targets small contiguous portions of the hCMV and H-2Ld genes, and produces a DNA fragment of 512 bp (FIG. 12e). The sense primer is directed at an hCMV intron sequence that is not present in the normal B6 genome. This eliminates the chance of inadvertent detection of other MHC class I genes that might occur if an H-21,d-specific PCR primer pair were used. This primer pair uses 30 cycles for arrays (94° C./60 sec, 62° C./60 sec, and 72° C./60 sec/cycle).

b. H-2Ld Gene Product Detection.

The H2Ld gene should not be expressed in any tissue without the tetracycline-controlled transactivator and tetracycline present. However, leaky transcription from the CMV minimal promoter is expected in multiple tissues. This leaky expression should be detectable at the mRNA level. Total RNA from liver, heart, kidney, muscle, and spleen tissues is isolated by the neutral-phenol extraction method. The contaminating DNA is removed by DNase digestion at 37° C. for 2 hr. Then the RNA is reverse transcripted into cDNA with the reverse-transcriptase. The cDNA is tested by PCR with the same primers used above for the H-2Ld gene screening.

### Example 7

### Tetracycline-controlled βgalactosidase Transgenic Mice (P2LacZ)

The P2LacZ line, a transgenic mouse that displays tetracycline transactivator-controlled β-galactosidase expression in all tissues is/was generated with the plasmid pTo1412 which contains the β-galactosidase gene driven by the tetracyline-controlled promoter in cassette that can be isolated by digestion with Xmn III/Xho I restriction endonucleases. This gene expression cassette is isolated, and purified by gel electrophoreses. Three pico-liter of these two purified DNA constructs, in a concentration of 3 ng/μl, are co-injected into the C57BL/6 embryo by microinjection with the identical technique as above.

a. Gene Screening.

Transgenic mice are screened by PCR as above. DNA from tail tissues is analyzed using PCR primer pairs that specifically target to the LacZ gene (sense, 5′-GCA GGG TGA AAC GCA GGT C-3′; ANTISENSE, 5′-CAT TTT CAA TCC GCA CCT CGC-3) and produce a DNA fragment of 237 bp (FIG. 12d). For PCR analyses in these primer pairs, 25 cycles are used (94° C./30 sec, 60° C./20 sec, and 72° C./60 sec/cycle).

b. β-galactosidase Product Detection.

Gene transcription is detected by the β-galactosidase activity analysis or β-galactosidase staining of histologic tissue specimens. Various tissues, such as liver, lung, heart, muscle, kidney, are harvested and fixed in 10% formalin for 3 hr. After Tissue Tek OCT (Miles, inc., Elkhart, Ind.) embedding, the tissues are sectioned 14 gm thick and stained with routine X-gal staining solution. The littermates of the LacZ-positive P2LacZ transgenic mice are used to prepare F1 mice.

Each of the four types of transgenic mice described in Examples 4-7, i.e. the transgenic mice designated PIE, PIM, P2Ld, and P2LacZ, are inbred 2 to 3 generations to produce homozygous transgenic lines. For each line, this requires the screening and selection protocol outlined below. First, the transgenic founders that are proven to be transgene-positive are outbred with normal, syngenic C57BL/6 mice to generate additional offspring that express the transgene. These offspring are screened by PCR to identify the transgene carrier mice, some of which will be sacrificed to test for relative levels of transgene mRNA and transgene protein expression. The goal is to identify lines with the highest levels of transgene expression. The transgene-positive littermates of mice with high transgene expression, which should express similar high levels of transgene function, are inbred for 2 to 3 generations to produce homozygous transgenic mouse lines. Routine PCR is used to identify and select transgenic mice during the process. When inbred lines have been obtained, Southern Blot is used to identify lines with the best transgene expression. These lines are tested for homozygosity by cross-breeding them with normal, non-transgenic mice. All litter mates from a homozygous transgene parent should be transgene positive, as detected by DNA screening. If some of the offspring are transgene negative, the line is not homozygous. In this way, four homozygous transgenic mouse lines, PIE, PIM, P2Ld and P2LacZ, are established.

### Example 8

### P2 Double Transgenic Mice

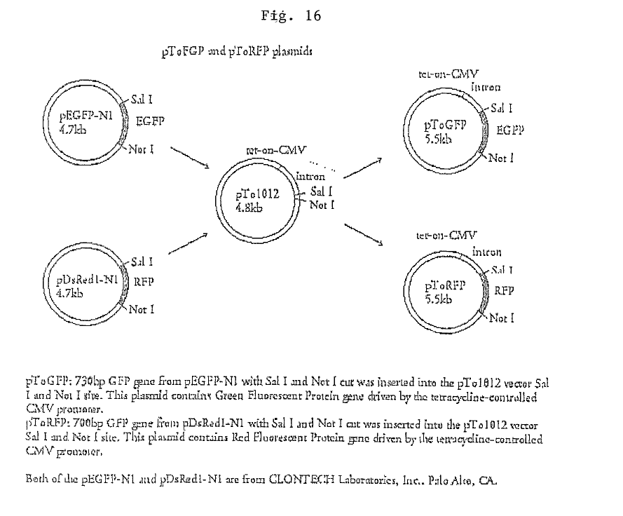

P2 double transgenic mice that express red fluorescence protein under the control of the tretracycline-controlled promoter, as well as the anti-sense RNA for the first intron of the CMV immediate-early gene were prepared. To make this transgenic line, C57BL/6 embryos were micro-injected with plasmids pToRFP and pVRBtA, respectively (See FIG. 16). Expression of the RFP gene product is detected by fluorescence histology of tissues from the transformed animals. Expression of the antisense RNA is assayed by RT-PCR as described above.

### Example 9

### Transgenic Mice Which Exhibit Tetracycline-controlled and Endothelial Specific Expression of P-galactosidase, (E-LacZ)

The endothelial-specific rtTA expression transgenic mice (PIE) is crossed with the β-galactosidase gene transgenic mice (P2LacZ mice) to produce the E-LacZ F1transgenic mice. In these mice, one set of chromosomes carries the PIE-derived rtTA/hCMV antisense genes and another set of chromosomes carrying the P2LacZ derived, transactivator-controlled LacZ gene. To validate this, tail tissues are collected and digested with 25 μl 0.1% collagenase at 37° C. for 2 hr. After a phenol and chloroform extraction to obtain DNA, 5 μl of DNA is analyzed by PCR with the rtTA hCMV antisense, and β-galactosidase primer pairs.

### Example 10

### Transgenic Mice Which Exhibit Tetracycline-controlled and Endothelial Specific H-2Ld Expression (E-Ld)

The endothelial-specific rtTA expression transgenic mice (PIE) are crossed with the H-2Ld transgenic mice (P2Ld) to produce the E-Ld F1 transgenic mice. In these mice, one set of chromosomes carries the PIE-derived rtTA/hCMV antisense genes, and the other set of chromosomes carries the P2Ld-derived transactivator-controlled H-2Ld gene. To evaluate this, tail tissues are collected, digested and tested by PCR with the rtTA, hCMV antisense, and CMV/H-2Ld primer pairs.

### Example 11

### Transgenic Mice Which Exhibit Tetracycline-controlled and Cardiac Myocyte Specific LaCZ Expression (M-LacZ)

The cardiac myocyte-specific rtTA gene expression transgenic mice (P-IM) is crossed with the β-galactosidase gene transgenic mice (P2LacZ mice) to produce the M-LacZ transgenic F1 mice. In these mice, one set of chromosomes carries the PIM-derived rtTA/hCMV antisense genes and the other set of chromosomes carries the P2LacZ-derived, transactivator-controlled LacZ gene. Each of these three genes, rtTA, hCMV antisense, and LacZ, should be detectable by PCR in the M-LacZ transgenic F 1 mice

### Example 12

### Transgenic Mice Which Exhibit Tetracycline-controlled and Cardiac Myocyte-specific H-2Ld Expression (M-Ld)

The myocyte-specific rtTA expression transgenic mice (PIM) are crossed with the H-2Ld transgenic mice (P2Ld ) to produce the M-Ld transgenic F1 mice. In these mice, one set of chromosomes carries the PIM-derived rtTA/hCMV antisense genes and the other set of chromosomes carries the P-2Ld-derived transactivator-controlled H2Ld gene. Tail tissues are collected, digested and tested by PCR with the rtTA, hCMV antisense primers, and CMV/H-2Ld primer pairs. All three genes, rtTA, antisense, and H-2Ld, should be carried by the M-Ld transgenic F1 mice.

As described above, the tetracycline-controlled promoter is associated with leaky gene expression that complicates the use of this promoter system for basic research or pharmaceutical application. β-galactosidase activity can be easily detected by in situ staining. The tetracycline-responsiveness and the tissue specificity of β-gal expression can also be easily tested by in situ tissue staining. In the absence of tetracycline the β-galactosidase should not be detected in any of tissues from either the E-LacZ or M-LacZ transgenic mice. This is tested by histochemistry in various tissues, such as liver, lung, heart, muscle, and kidney. To do this, the tissues is harvested from the transgenic F1 mice and fixed in 10% formalin for 3 hr, embedded in Tissue Tek OCT (Miles, inc., Elkhart, Ind.), sectioned (14 μm), and stained with routine X-gal staining solution. Tissues from non-transgenic animals serve as controls.

A quantitative analysis of P-galactosidase by ONPG colorimetric assay can also be used for this study. ONPG is a chromogenic substrate for the enzyme β-galactosidase. Total protein will be extracted from the various tissues with PBS buffer, incubated with the ONPG for 30 min. at 37° C. β-galactosidase activity is quantitated by spectrophotometric absorption at 450 nm.

Both methods of β-gal detection have advantages and disadvantages. X-gal staining of histologic sections is sensitive, and allows the localized detection of (β-gal production within the tissues, but X-gal staining is essentially a qualitative test. X-gal staining is utilized to detect background levels of gene expression and for detection of tissue-specific β-gal production. In contrast, the ONPG colorimetric assay is quantitative, but does not permit analysis of tissue specific β-gal expression. The ONPG assay is complicated by the fact that most tissues have background absorption at 450 nm, so matched tissues from non-transgenic mice must be used to establish baseline absorption values. This technique is primarily utilized for the general quantitation of β-gal expression in murine tissues.

The tissue specific expression of the β-galactosidase gene should be turned on when tetracycline is administrated to E-LacZ or MLacZ mice. To test this, tetracycline is given drinking water or via subcutaneous pellet. In initial studies, tetracycline or doxycycline (2 mg/ml) is administrated via drinking water for 48 hours before the tissue harvesting. Thereafter, various tissues are collected and tested for β-galactosidase.

The leakiness, tetracycline-inducibility, and tissue-specificity of exogene expansion is also evaluated in the E-Ld and M-Ld transgenic F1 mice. In this situation, the expression of the H-2Ld exogene product is detected by immunohistochemistry or Western Blot analysis. Again, the F1 mice are either treated with tetracycline or not via the drinking water or subcutaneously implanted pellets as described above. The tissue specific expression of Ld protein is detected in different tissues by immunohistochemistry with the specific anti-H-2Ld antibody. Briefly, various tissues from the transgenic F1 mice, are harvested, embedded and sectioned in 5 μm. A biotin-labeled mouse anti-Ld monoclonal antibody 30-5-7 (American Type Culture Collection, Rockville, Md.) is used as the primary antibody for the L^{d} protein detection, and HRP conjugated aviden is used for the colorogenic reaction.

Western Blotting is also used for the L^{d} protein detection. Various tissues, such as liver, lung, heart, kidney, muscle, and spleen, are harvested, homogenized, and treated with the SDS PAGE sample buffer at 100° C. for 5 minutes. The total proteins is separated by PAGE and blotted into a PVDF membranes where the Ld protein can be specifically detected with the mouse anti-L^{d}mAb.

What is claimed is:

1. A method for preparing a transgenic animal having controllable tissue specific expression of an exogenous gene comprising:
a) providing a first transgenic animal whose genome comprises an F transgene which comprises the exogenous gene operably linked to an F promoter that is upregulated by a transactivator protein;
b) providing a second transgenic animal whose genome comprises
(i) an S transgene which comprises an antisense gene operably linked to an S promoter that is downregulated by said transactivator protein, wherein the antisense gene encodes a transcript which inhibits processing of the F transgene transcript; and
(ii) a T transgene comprising a tissue specific promoter operably linked to a gene encoding said transactivator protein; and
c) breeding the first transgenic animal with the second transgenic animal to provide a transgenic offspring animal whose genome comprises the F transgene, the S transgene, and the T transgene.

2. The method of claim 1 wherein the first transgenic animal, the second transgenic animal, and the transgenic offspring animal are mice.

3. The method of claim 1 wherein the antisense gene encodes an RNA whose sequence is complementary to a region in the F promoter.

4. The method of claim 1 wherein the F promoter is a tetracycline controlled promoter which comprises the operator sequence of the E. coli tetracycline resistance gene fused to the minimal promoter sequence of the cytomegalovirus immediate early gene;
wherein the transactivator protein is the reverse tetracycline controlled transactivator;
and wherein the antisense gene encodes a transcript which binds to the minimal promoter sequence of the cytomegalovirus immediate early gene.

5. The method of claim 1 wherein the genome of the first transgenic animal further comprises a transgene comprising a constitutive promoter operably linked to a gene encoding said transactivator protein.

6. A system for controlling expression of an exogenous gene in specific tissues of a transgenic animal comprising,
(a) an F transgene comprising an F promoter that is up-regulated by a transactivator protein and multiple restriction sites for subcloning an exogene, and
(b) a transgenic animal whose genome comprises
(i) an S transgene which comprises an antisense gene operatively linked to an S promoter that is downregulated by said transactivator protein, wherein the antisense gene encodes a transcript which inhibits processing of the F transgene transcript; and
(ii) a T transgene comprising a tissue specific promoter operatively linked to a gene encoding said transactivator protein.

7. The system of claim 6 wherein said F transgene further comprises an exogene which is operatively linked to said F promoter.

8. The system of claim 6 wherein the F promoter is a tetracycline controlled promoter which comprises the operator sequence of the E. coli tetracycline resistance gene fused to the minimal promoter sequence of the cytomegalovirus immediate early gene.

9. The system of claim 8 wherein the antisense gene of the S transgene encodes a transcript which binds to the minimal promoter sequence of the cytomegalovirus immediate early gene.

10. The system of claim 8 wherein the antisense gene of the S transgene encodes a transcript which inhibits splicing of the F transgene transcript.

11. A transgenic animal whose genome comprises
(a) an first transgene which comprises an antisense gene operably linked to an S promoter that is downregulated by a transactivator protein, wherein the antisense gene encodes a transcript which is complementary to a region of a constitutive promoter; and
(b) a second transgene comprising a tissue specific promoter operably linked to a gene encoding said transactivator protein.

12. The transgenic animal of claim 11 wherein the antisense gene encodes a transcript which binds to a region selected from the group consisting of the 5′ untranslated region of the CMV immediate early gene, the first intron of the CMV immediate early gene, and combinations thereof.

13. The transgenic animal of claim 11 wherein the transactivator protein is the reverse tetracycline-controlled activator.

14. The transgenic animal of claim 11 wherein the tissue specific promoter is an endothelial tissue specific promoter or a cardiac myocyte specific promoter.

15. The transgenic animal of claim 11 wherein the antisense gene encodes an RNA that prevents splicing of a gene transcript.

16. The transgenic animal of claim 11 wherein the animal is homozygous for the first transgene and the second transgene.

17. The transgenic animal of claim 11 wherein the first transgene comprises a constitutive promoter and a tetracycline operator sequence, wherein the tetracycline operator sequence is linked to the 3′ end of the constitutive promoter and the 5′ end of the antisense gene.

18. A DNA construct comprising a transgene which comprises an antisense gene operably linked to an S promoter that is downregulated by a transactivator protein, wherein the antisense gene encodes a transcript which is complementary to a region of a constitutive promoter.

19. The DNA construct of claim 18 wherein the antisense gene encodes a transcript which binds to a region selected from the group consisting of the 5′ untranslated region of the CMV immediate early gene, the first intron of the CMV immediate early gene, and combinations thereof.

20. The DNA construct of claim 18 wherein the transgene comprises a constitutive promoter and a tetracycline operator sequence, wherein the tetracycline operator sequence is linked to the 3′ end of the constitutive promoter and the 5′ end of the antisense gene.
